(19) United States
(12) Patent Application Publication
(10) Pub. No.: US 2004/0044079 A1
(43) Pub. Date: Mar. 4, 2004

(54) Methods and compositions for weight control

(75) Inventors: Gregory Dean Sunvold, Eaton, OH (US); Robert Jason Vickers, Dayton, OH (US); Gary Robert Kelm, Cincinnati, OH (US); Susan Liew Giovengo, Mason, OH (US); Steven Trevor Meller, Loveland, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The IAMS Company

(21) Appl. No.: 10/654,329

(22) Filed: Sep. 3, 2003

Related U.S. Application Data
(60) Provisional application No. 60/408,170, filed on Sep. 4, 2002.

(51) Int. Cl. A61K 31/202; A23K 1/165; A23K 1/17

(52) U.S. Cl. 514/560; 514/558; 424/442

(57) ABSTRACT

Disclosed herein are methods of promoting weight control in a companion animal comprising orally administering one or more non-glyceryl derivatives of C₁₇ or greater fatty acids. Also disclosed are methods for promoting weight control in a human comprising orally administering non-glyceryl derivatives of C₁₇ or greater fatty acids, wherein the fatty acid derivatives do not cause the human to reduce food consumption. Further disclosed are methods for promoting weight control in a human or companion animal comprising orally administering lotus leaf extract.

Further disclosed are dietary compositions for promoting weight control in a companion animal., wherein such compositions comprise one or more of the non-glyceryl derivatives of C₁₇ or greater fatty acids and the lotus leaf extract.

### CROSS REFERENCE TO RELATED APPLICATION

This application claims the benefit of U.S. Provisional Application No. 60/408,170, filed on Sep. 4, 2002.

### BACKGROUND OF THE INVENTION

Obesity is a major health problem for both humans and companion animals. A large percentage of dogs and cats receiving veterinary care are overweight. Obesity in dogs and cats is linked to many health problems, including diabetes, osteoarthritis, and cardiopulmonary diseases. Obesity in humans is linked to the same and other problems. Therefore it is important to maintain healthy weight in humans and companion animals in order to minimize disease risk.

A number of approaches have been proposed or used to help mammals lose weight. These include agents that interfere with digestion or absorption of nutrients, agents that induce a sense of satiety, thus reducing food intake, and agents that accelerate metabolic rate.

One approach that has been tried is the introduction of nutrients directly into the small intestine in an attempt to reduce concomitant food intake. This approach is believed to function via interaction of the nutrients with putative receptors in the small intestine, particularly in the distal small intestine (jejunum, ileum) that are believed to participate in the natural mechanisms that induce termination of a meal. Some of these studies employed lipids. For example, see Welch, I. et al., Gastroenterology 89,1293 (1985) and Welch et al., Gut 29, 306 (1988); Walls, E. K., and Koopmans, H. S., Physiology and Behavior 45, 1223 (1989); Woltman, T. et al., Am. J. Physiology 269, R7 (1995); Woltman, T., et al., Am. J. Physiology 269, R1420 (1995); Greenberg, D., et al., Am. J. Physiology 259, R110 (1990); Greenberg, D., et al, Am. J. Physiology 264, R409 (1993); Greenberg, D., Appetite 31, 229 (1998); Greenberg, D., pp. 40-70, in Satiation from Gut to Brain, G. P. Smith (ed.), Oxford Univ. Press, New York (1998); Meyer, J. H., et al., Am. J. Physiology 275, R1293 (1998); and Meyer et al. Am. J. Physiology 275, R1308 (1998).

However, such methods have limited practical use for weight control in humans and companion animals, because of the requirement for catheter infusion. This ordinarily cannot be done at home, and greatly increases the cost and inconvenience of the procedure. Catheter infusion into the gastrointestinal tract is also undesirable because it is uncomfortable and requires an unusual technique for delivery of nutrients. Reducing weight by inducing a sense of fullness could also be problematic if reduced food intake leads to nutrient deficiencies.

None of the above studies addressed whether ingested fats, fatty acids, or fatty acid derivatives reduce food intake or otherwise induce weight loss. Cairns et al. (U.S. Pat. No. 6,054,480), however, has reported that incorporation of myristic acid, a saturated C₁₄ fatty acid, into a high fat diet fed to mice caused weight loss or reduction of weight gain in the mice.

Despite the above reports, there currently remains a need for methods of inducing weight loss in companion animals and humans.

### SUMMARY OF THE INVENTION

The present invention provides a method for promoting weight control in a companion animal comprising orally administering one or more non-glyceryl derivatives of a C₁₇ or greater fatty acid.

The present invention also provides a method for promoting weight loss or weight control in a human comprising orally administering a non-glyceryl derivative of a C₁₇ or greater fatty acid, wherein the fatty acid derivative does not cause the subject to reduce food consumption.

The present invention also provides a dietary composition for promoting weight control in companion animals, the composition comprising, on a dry matter basis by weight, at least about 0.5% of a non-glyceryl fatty acid derivative of a C₁₇ or greater saturated or monounsaturated fatty acid, about 15% to about 55% protein, and about 9% to about 35% dietary fat.

The present invention also provides a method for promoting weight control in companion animal comprising orally administering lotus leaf extract.

The present invention also provides a dietary composition for promoting weight control in a companion animal, the composition comprising, on a dry matter basis, at least about 0.05% lotus leaf extract.

The present invention also provides a method for promoting weight control in a human comprising orally administering lotus leaf extract, wherein the lotus leaf extract does not cause the human to reduce food consumption.

An advantage of the invention is that in general the fatty acid derivatives and the lotus leaf extract do not cause the companion animal to reduce its food consumption. This is important because a reduction in food intake can cause inadequate nutrient intake. Inadequate nutrient intake increases the risk for many diseases in humans and companion animals, including cancer, cardiovascular disease, and arthritis. It also reduces resistance to infection.

Another advantage of the invention is that the fatty acid derivatives and lotus leaf extract cause weight loss or a reduction in weight gain when used in combination with a low-fat or normal fat content diet. This is important because when humans want to control their weight or the weight of their companion animals, they will ordinarily avoid a high-fat diet and preferably use a low-fat diet.

Another advantage is that according to the methods of the invention the fatty acid derivatives can be ingested, avoiding the cost, inconvenience, and discomfort of infusion into the intestine. A further advantage is the use of derivatives of C₁₇ or greater fatty acids, including the C₁₈ fatty acids, which are more abundant in nature than C₁₂ to C₁₅ fatty acids.

These and other features, aspects, and advantages of the invention will become better understood with reference to the following drawings, description, and appended claims.

### BRIEF DESCRIPTION OF THE DRAWINGS

FIG. 1 shows the average total daily food intake per dog by week during the study (Example 1).

FIG. 2 shows the average food intake in grams per kg body weight per day (Example 1).

FIG. 3 shows the percent change in body weight after the start of the experimental period (Example 1).

FIG. 4 shows the average body weight of the dogs by week during the study (Example 1).

### DETAILED DESCRIPTION OF THE INVENTION

It has surprisingly been found that ingestion by dogs of food in which some of the fat was replaced with free fatty acids resulted in greater weight loss compared to ingestion of food with the identical content except that all of the fat was in the form of triacylglycerol esters of the fatty acids. Greater weight loss was also achieved with food containing non-glyceryl esters of fatty acids. These weight loss effects were achieved without reducing food intake. It is expected that the same effects would be achieved in other companion animals, including cats.

It has also surprisingly been found that feeding dogs a low calorie dog food supplemented with lotus leaf extract results in the same weight loss as feeding them the low calorie food alone, and moreover results in greater loss of fat and greater gain of lean body mass. It is expected that the same effects would be achieved in other companion animals, including cats, as well as in humans.

As used herein, the term “companion animal” includes dogs, cats, and other mammals commonly kept by humans for companionship.

As used herein, the term “reduced food consumption” or “reduced food intake” refers to a reduction in the dry mass or caloric content of food consumed.

As used herein, the term “lean body mass” refers to the mass of all the components of a human or companion animal, including muscle and bone, except for fat.

As used herein, the term “dried beet pulp” includes dried beet pulp in which the sugar has been removed.

As used herein, the term “brewer's yeast” includes dried brewer's yeast.

As used herein, the term “fatty acids” refers to linear or branched hydrocarbons terminating in a single carboxyl group. Fatty acids of the invention preferably contain from 17 to about 30 carbon atoms, more preferably from 17 to about 24 carbon atoms. In a specific embodiment, the fatty acids contain 18 carbon atoms. The carbon chain can be branched or linear. Preferred fatty acids have a linear carbon chain. The carbon chain can be saturated, monounsaturated, or polyunsaturated, and typically has from 0 to about 6, preferably from 0 to about 5, more preferably from 0 to about 3 carbon-carbon double bonds. The carbon chain can also have one or two hydroxyl groups, as in ricinoleic acid. Preferred fatty acids have no hydroxyl groups. Preferred fatty acids are selected from the group consisting of heptadecanoic acid, stearic acid, arachidic acid, behenic acid, lignoceric acid, cerotic acid, oleic acid, cis-11-octadecenoic acid, linoleic acid, alpha-linolenic acid, gamma-linolenic acid, eicosapentaenoic acid, docasahexaenoic acid, arachidonic acid, erucic acid, and mixtures thereof. Especially preferred fatty acids are selected from the group consisting of oleic acid, linoleic acid, and mixtures thereof.

In specific embodiments of the invention, the fatty acid derivatives comprise the fatty acids derivable from plant oils, including corn oil, soybean oil, canola oil, flax oil, sunflower oil, safflower oil, rapeseed oil, cottonseed oil, coconut oil, linseed oil, palm oil, olive oil, and mixtures thereof. In other specific embodiments of the invention, the fatty acid derivatives comprise the fatty acids derivable from animal fats, other animal by-products, and mixtures thereof. Fatty acids for use in the invention can also be derived from plant protein meals, including flax seed meal, rapeseed meal, cottonseed meal, linseed meal, soybean meals, and the like.

In specific embodiments of the invention, the fatty acid derivatives comprise mixtures of fatty acids derived by hydrolysis of vegetable oil, including corn oil, soybean oil, canola oil, flax oil, sunflower oil, safflower oil, rapeseed oil, cottonseed oil, coconut oil, linseed oil, palm oil, olive oil, and mixtures thereof. In other specific embodiments of the invention, the fatty acid derivatives comprise mixtures of fatty acids derived by hydrolysis of animal-based fats in general, from poultry fat, including duck, goose, turkey, and chicken fats, from chicken by-product meal or whole ground chicken, from eggs or dried whole eggs, from lamb, lamb meal, beef, beef by-product meal, or fish; and from mixtures thereof. In other specific embodiments, the fatty acid derivatives comprise fatty acids derived by hydrolysis of dairy fat, including cream and butter oil.

Chicken fat can be a good source of free fatty acids. Suitably preserved with antioxidants, chicken fat can contain from 0% to about 15% free fatty acids as a percentage by weight of total fat.

As used herein, the term “dietary fat” includes monoacyl, diacyl, and triacyl glycerides, as well as the non-glyceryl fatty acid derivatives.

As used herein, the term “non-glyceryl fatty acid derivatives” refers to free fatty acids and alkyl esters of such fatty acids. In one embodiment, the fatty acid derivatives are C₁-C₆ alkyl fatty acid esters. Preferred fatty acid derivatives are methyl, ethyl, n-propyl, isopropyl, n-butyl, and isobutyl esters, and mixtures thereof. Especially preferred are ethyl esters. Especially preferred ethyl esters are ethyl oleate, ethyl linoleate, and mixtures thereof.

As used herein, the term “weight control” includes weight loss, weight maintenance, and a reduction in weight gain. As used herein, the term “weight control” also includes loss of fat tissue, maintenance of fat tissue, and a reduction in the gain of fat tissue. When orally administered in suitable amounts to humans or companion animals, the non-glyceryl fatty acid derivatives described herein or lotus leaf extract will promote weight loss, fat loss, or both. The companion animal or human will experience weight or fat loss, weight or fat maintenance, or a reduction in weight or fat gain versus a subject eating a comparable diet lacking the non-glyceryl fatty acid derivatives or lotus leaf extract.

To promote weight control in a companion animal or human, the one or more non-glyceryl derivatives of C₁₇ or greater fatty acids can be administered with the subject's food or meal. When administered with the food or meal, the derivatives can be incorporated into the food, or can be administered separately (i.e., as a supplement), as for instance in a pill, prior to, during, or after consumption of a meal. Preferably, the administration is within 90 minutes of a meal, more preferably within 60 minutes of a meal, more preferably still within 30 minutes of a meal.

In one specific embodiment of administration of the fatty acid derivatives with a meal, the meal comprises, on a dry matter basis by weight, at least about 0.5% non-glyceryl derivatives of C₁₇ or greater fatty acids, about 15% to about 55% protein, and about 9% to about 35% dietary fat. In another specific embodiment, the meal comprises the same ranges of protein and fat, and at least about 1%, about 1% to about 13%, about 1% to about 5%, at least about 2%, about 2% to about 4%, at least about 3%, or about 3% to about 8% non-glyceryl derivatives of C₁₇ or greater fatty acids.

In one specific embodiment of a method for promoting weight control, the ester derivatives are hydrolyzable in the gastro-intestinal tract of the human or companion animal subject, so as to release the corresponding free fatty acids. The fatty acid esters can be hydrolyzed anywhere in the gastrointestinal tract, including the mouth, esophagus, stomach, small intestine, or large intestine.

In specific embodiments, at least about 100 mg, at least about 300 mg, or at least about 500 mg of non-glyceryl fatty acid derivatives is administered orally per kg body weight per day. In other specific embodiments, about 100 mg to about 2 g, about 200 mg to about 1.5 g, or about 400 mg to about 900 mg of non-glyceryl fatty acid derivatives is administered orally per kg body weight per day.

One of the advantages of a method for promoting weight loss or weight control in a human, dog, or cat comprising orally administering non-glyceryl fatty acid derivatives is that the fatty acid derivatives do not generally cause the subject to reduce food consumption. In a specific embodiment of this method applied to humans, the administration is with a meal. In another specific embodiment, the non-glyceryl fatty acid derivatives are free fatty acids.

Another embodiment of the invention is a dietary composition for promoting weight control in a companion animal comprising, on a dry matter basis by weight, at least about 0.5% non-glyceryl derivatives of C₁₇ or greater saturated or monounsaturated fatty acids, about 15% to about 55% protein, and about 9% to about 35% dietary fat. In a specific embodiment, the composition comprises about 1% to about 13% non-glyceryl fatty acid derivatives. In another specific embodiment, the composition comprises about 3% to about 8% non-glyceryl fatty acid derivatives. In other specific embodiments, the composition comprises at least about 3% or at least about 8% non-glyceryl fatty acid derivatives.

In a specific embodiment of the dietary composition for promoting weight control in a companion animal, the fatty acid derivatives are free fatty acids. In another specific embodiment, the fatty acid derivatives comprise non-glyceryl esters.

In a specific embodiment of the composition, the fatty acids in the fatty acid derivatives are selected from the group consisting of heptadecanoic acid, stearic acid, arachidic acid, behenic acid, lignoceric acid, cerotic acid, oleic acid, cis-11-octadecenoic acid, erucic acid, and mixtures thereof.

In a specific embodiment of the composition, the non-glyceryl fatty acid ester is ethyl oleate.

In a specific embodiment, the invention provides a dietary composition for promoting weight control in a companion animal comprising, on a dry matter basis by weight, at least about 0.5% non-glyceryl derivatives of C₁₇ or greater saturated or monounsaturated fatty acids, about 15% to about 55% protein, and about 9% to about 35% dietary fat. In one particular embodiment, this composition includes corn meal, ground whole grain sorghum, chicken by-products, ground whole grain barley, fish meal, chicken fat, dried beet pulp, dried egg product, brewer's yeast, and a meat selected from the group consisting of chicken, beef, and lamb.

In another specific embodiment, the composition includes chicken, brewer's rice, fish meal, dried egg product, dried beet pulp, and flax meal.

In another specific embodiment, the composition includes dried beet pulp, dried egg products, fish meal, and chicken by-products.

In another specific embodiment, the composition includes chicken by-products; fish meal; chicken fat; dried beet pulp; dried egg product; a source of protein selected from the group consisting of chicken and lamb; and a source of carbohydrate selected from the group consisting of corn grits, corn meal, and ground corn grits.

In another specific embodiment, the composition includes chicken by-products or beef byproducts; dried egg product; brewer's yeast; and a source of protein selected from the group consisting of chicken, turkey, beef, and fish.

Companion animal food compositions generally contain protein, fat, carbohydrate, fiber, vitamins, and minerals. In particular embodiments of a dietary composition for promoting weight control with the non-glyceryl fatty acid derivatives in a companion animal, the composition includes certain compositions of protein, fat, carbohydrate, and fiber. Specific embodiments of the compositions comprise, for instance, about 15% to about 55%, about 19% to about 36%, about 30% to about 55%, or about 25% protein. Other specific embodiments comprise about 4% to about 35%, about 9% to about 35%, about 4% to about 26%, about 9% to about 26%, about 13% to about 20% or about 15% dietary fat. Other specific embodiments comprise about 1% to about 26%, about 2% to about 10%, or about 4% crude fiber. The above percentages are on a dry matter basis by weight.

The source of protein for the compositions of the invention can be, for example, selected from the group consisting of turkey, beef, chicken, lamb, turkey by-products, beef by-products, chicken by-products, lamb by-products, liver, hearts, kidneys, soybean meal, soybean concentrate, soy protein isolate, corn gluten meal, eggs, and fish. Protein can also be provided by flax seed meal, rapeseed meal, cottonseed meal, and linseed meal.

The source of fat in specific embodiments, for example, can be selected from the group consisting of tallow, poultry fat, chicken fat, turkey fat, beef fat, soy oil, and corn oil, and mixtures thereof.

The source of carbohydrate in specific embodiments of the compositions of the invention, for example, can be selected from the group consisting of corn, corn grits, wheat, rice, oats, grain sorghum, barley, amaranth, corn by-products, wheat by-products, rice by-products, oat by-products, grain sorghum by-products, barley by-products, amaranth by-products, and mixtures thereof.

The above lists of sources of protein, fat, and carbohydrate are exemplary and are not intended to exclude other possible sources.

The dietary compositions for companion animals will typically be solid food. Solid food can be moist (e.g., canned). Typically moist food will have up to 80% by weight water. Solid food can also be dry. Dry food can be in the form of kibble. It will typically have 12% or less, or preferably 10% or less water. Solid foods can also be semi-moist, having intermediate levels of moisture. Semi-moist foods can have, for instance, 20-60%, 30-50%, 40-50%, or about 45% moisture.

The present invention also provides for food compositions for humans comprising non-glyceryl derivatives of C₁₇ or greater fatty acids, or non-glyceryl derivatives of saturated or monounsaturated C₁₇ or greater fatty acids, or lotus leaf extract. The fatty acid derivatives or lotus leaf extract can be added, in amounts sufficient to promote weight control, to fattening food items, such as high fat or high sugar foods. These include beverages, frozen desserts, sauces, snacks, and cereals. For instance, they can be incorporated into french fries, hamburgers, hot dogs, ice cream, pastries, cakes, cookies, and candy bars.

In a specific embodiment of a method for promoting weight control in a companion animal comprising administering lotus leaf extract, the administration of lotus leaf extract promotes a decrease or maintenance of fat and an increase or maintenance of lean body mass in the companion animal. In another specific embodiment, the lotus leaf extract does not cause the companion animal to reduce food consumption. As used herein, the term “lotus leaf extract” refers to a solvent extract of lotus leaves (Nelumbo nucifera), e.g., an ethanol extract. The term also includes whole lotus leaves or any composition that includes a crude extract from lotus leaves. Where weight percentages are used, this refers to a 20:1 concentrated extract of lotus leaves. If a less or more concentrated extract is used, appropriate conversion should be made. For instance, where a composition is described containing 0.3% lotus leaf extract, this refers to 0.3% of a 20:1 extract. This would be equivalent to a composition containing 0.6% of a 10:1 extract. Lotus leaf extract is available commercially from, for instance, Advanced Herbal Ingredient Group, Inc., 6# Lixiang Rd., Changsha High & New Tech, Development Zone, China, Changsha, China; phone 86-731-4021303/86-13507475971. Lotus leaf extract has been used in Chinese medicine to facilitate removal of free radicals from the system.

In specific embodiments of a method for promoting weight control in a companion animal comprising administering lotus leaf extract, the companion animal is a cat or a dog.

To promote weight control in a companion animal or human, the lotus leaf extract can be administered with the subject's food or meal. When administered with the food or meal, the derivatives can be incorporated into the food, or can be administered separately (i.e., as a supplement), as for instance in a pill, prior to, during, or after consumption of a meal. Preferably, the administration is within 90 minutes of a meal, more preferably within 60 minutes of a meal, more preferably still within 30 minutes of a meal.

In specific embodiments of the methods for using lotus leaf extract to promote weight control in a companion animal or human, comprising administering lotus leaf extract with the subject's food or meal, the meal comprises, on a dry matter basis by weight, at least about 0.05%, at least about 0.1%, about 0.1% to about 2%, at least about 0.2%, about 0.2% to about 0.6%, or at least about 0.3% lotus leaf extract. In a specific embodiment of these methods, the meal comprises, on a dry matter basis by weight, about 15% to about 55% protein and about 9% to about 35% dietary fat.

In specific embodiments of the methods to promote weight control in a companion animal or human, at least about 10 mg, at least about 20 mg, about 20 mg to about 150 mg, at least about 30 mg, about 30 mg to about 100 mg, or at least about 50 mg of lotus leaf extract is administered per kg body weight per day.

Another embodiment of the invention is a dietary composition for promoting weight control in a companion animal, the composition comprising, on a dry matter basis by weight, at least about 0.05% lotus leaf extract. This composition can comprise chicken by-products or beef by-products; dried egg product; and a source of protein selected from the group consisting of chicken, lamb, turkey, beef, and fish.

In a specific embodiment, the composition with 0.05% lotus leaf extract comprises corn meal, ground whole grain sorghum, chicken by-product meal, ground whole grain barley, fish meal, chicken fat, dried beet pulp, brewer's yeast, dried egg product, and a source of protein selected from the group consisting of chicken, beef, and lamb.

In another specific embodiment, the composition with 0.05% lotus leaf extract comprises chicken by-products, dried egg product, chicken, brewer's rice, fish meal, dried beet pulp, and flax meal.

In another specific embodiment, the composition with 0.05% lotus leaf extract comprises dried egg product, dried beet pulp, fish meal, chicken by-products, and a meat selected from the group consisting of chicken, lamb, turkey, beef, and fish.

In another specific embodiment, the composition with 0.05% lotus leaf extract comprises chicken by-products; fish meal; chicken fat; dried beet pulp; dried egg product; a source of protein selected from the group consisting of chicken and lamb; and a source of carbohydrate selected from the group consisting of corn grits, corn meal, and ground corn grits.

In another specific embodiment, the composition with 0.05% lotus leaf extract comprises brewer's yeast; chicken by-products or beef by-products; dried egg product; and a source of protein selected from the group consisting of chicken, lamb, turkey, beef, and fish.

In particular embodiments, the lotus leaf dietary compositions for promoting weight control in a companion animal comprise, on a dry matter basis by weight, about 0.1% to about 2% lotus leaf extract.

The lotus leaf dietary compositions preferably are solid food compositions. The solid food can be moist, semi-moist, or dry. The dry compositions in one embodiment are dry kibble.

In a method for promoting weight control in a human comprising orally administering lotus leaf extract, the lotus leaf extract does not cause the human to reduce food consumption. In a specific embodiment of this method, the administration of the lotus leaf extract promotes a decrease or maintenance of fat and an increase or maintenance of lean body mass in the human.

The invention can be better understood with reference to following example.

### EXAMPLE 1

Materials and Methods.

In phase I of the study, 80 beagle and mongrel dogs were fed a weight gain diet (36% protein, 26% fat by dry weight). The dogs were allowed to eat as much as they wished for 16 weeks. Weekly weight gain and daily food intake were measured for each dog.

In phase II, the 20 dogs that were the poorest weight gainers were eliminated from the study. The 60 remaining dogs were randomly assigned to one of the four treatment groups. The treatment diets were the following:

Control. Similar to EUKANUBA REDUCED FAT dry dog food, consisting of approximately 19% protein and 10% fat on a dry weight basis, with an additional 3% poultry fat to achieve a total fat content of 13%.

Free fatty acid mix. Identical to the control diet, but with 3% free fatty acids in place of the 3% poultry fat. The free fatty acid was technical grade linoleic acid.

Ethyl esters of fatty acid mix. Identical to control diet, but with 3% ethyl ester of fatty acid in place of the 3% poultry fat. The ethyl ester used was ethyl oleate.

Lotus leaf extract. Identical to the control, but with an additional 0.3% extract of lotus leaf. Lotus leaf extract has been used in China to promote human weight loss. It is purported to help induce a sense of satiety and reduce food consumption.

The dogs were given the treatment diets for 12 weeks. During this time the dogs were allowed to eat as much as they wished. Daily food intake and weekly body weight were measured. Body composition measurements were also taken, including fat, lean body mass, bone mineral content (BMC), and bone mineral density (BMD). These measurements help to determine the cause of weight loss—for instance, whether it is due to an increase in satiety and therefore reduced food intake, or an increase in thermogenesis. The measurements also allow a determination of whether weight loss results from a reduction in lean body mass or a loss of fat, or a combination of both.

Results

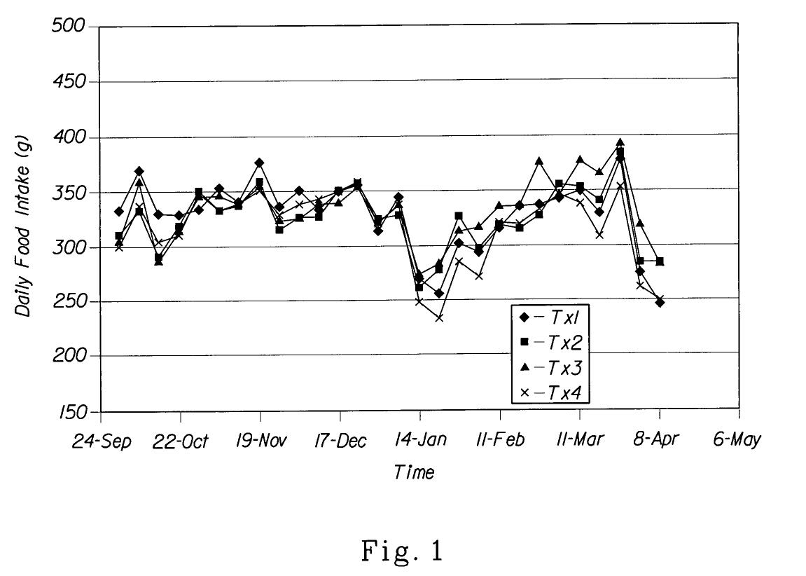

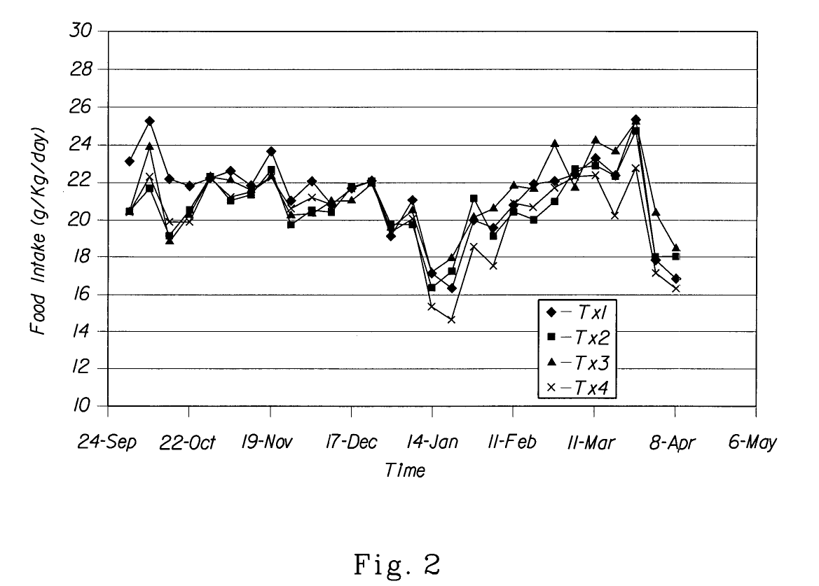

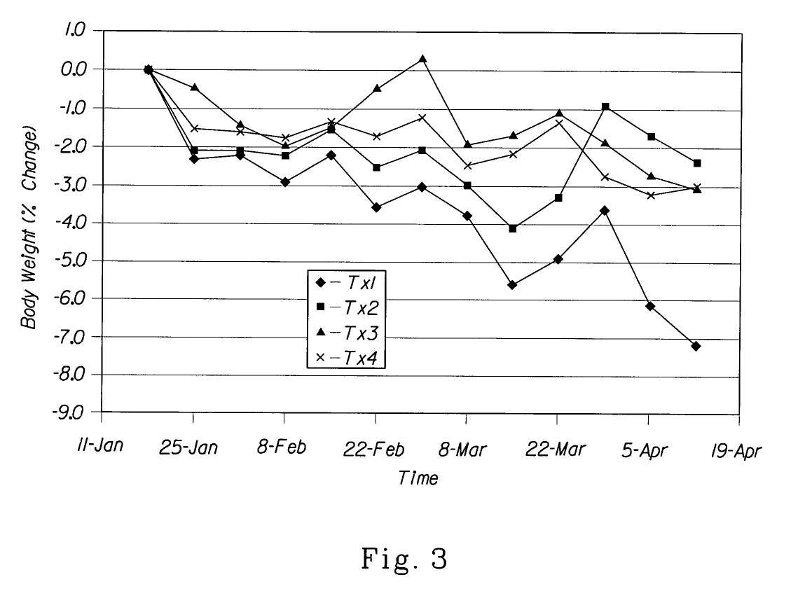

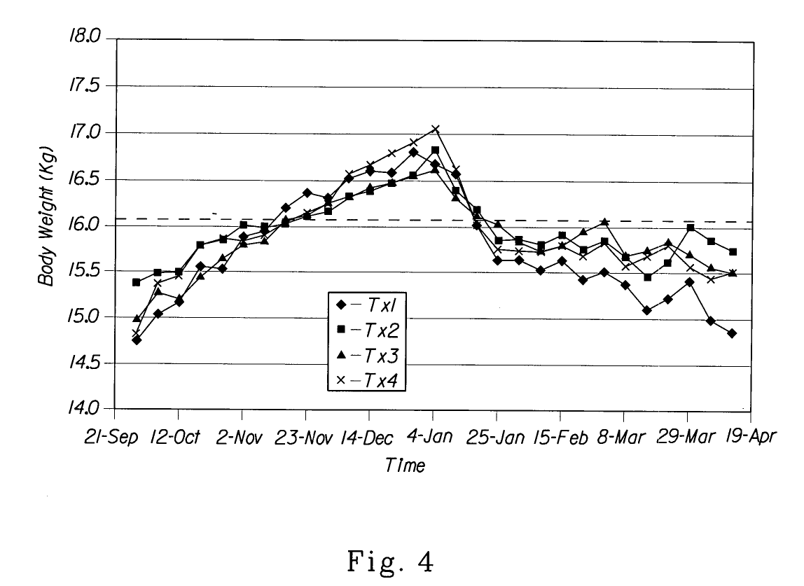

The results of the measurements of food intake and body weight are shown in FIGS. 1-4. FIGS. 1 and 2 show that during the conditioning period of phase I, average food intake was about the same for all four groups, except that during the first few weeks, the dogs later assigned to group 1, free fatty acid mix, ate slightly more. During the experimental period of phase II, the dogs eating the control diet ate the least. The dogs eating the diet with lotus leaf extract generally ate the most. The dogs eating the free fatty acid or ethyl ester of fatty acid diets ate intermediate amounts, slightly more than the control diet. FIGS. 3 and 4 show that weight loss was generally greatest for the dogs eating the free fatty acid diet. This is despite the fact that these dogs ate more food than the dogs in the control group. The dogs eating the ethyl ester diet also lost more weight than the control group for the first 9 weeks of phase II. In the 10th-12th weeks of phase II their average weight increased above that of the control group. But the increased weight loss of the dogs eating ethyl esters of fatty acids compared to the control group for most of the study is remarkable given that they also ate slightly more food than the dogs eating the control diet. The conclusion of the study is that the diet containing free fatty acids increased weight loss the most over a control diet containing a conventional fat mix, where nearly all fats are in the form of glycerol esters. This is despite the fact that the dogs eating the free fatty acid diet ate slightly more than those on the control diet. A diet with ethyl esters of free fatty acids also appeared to induce weight loss for most of the experimental period, despite not decreasing and perhaps slightly increasing food consumption compared to the control diet.

It is expected that similar effects would be obtained with cats, horses, and humans.

At the beginning and end of phase II, several body composition and body condition measurements were taken, with the results shown in Tables 1-5. FIGS. 1 and 2 show that the dogs eating the lotus leaf extract diet generally ate the most. Yet despite the greater food consumption, dogs eating the lotus leaf extract had about the same weight loss in phase II as the dogs eating the control diet (FIGS. 3 and 4). Furthermore, the data of Table 4 show that the dogs eating the lotus leaf diet had a greater loss of fat than dogs eating the control diet, while having a greater increase in lean body mass than the dogs of any of the other three groups. Thus, the lotus leaf extract did not induce a decrease in food consumption compared to the control diet, but did induce a loss of fat and increase in lean body mass.

TABLE 1
Description of Body Condition Scores

| Score | Condition | Score Description |
|---|---|---|
| 1 | Thin | Ribs, lumbar vertebrae and pelvic bones easily visi- |
| | | ble. No palpable fat in flank folds, folds often ab- |
| | | sent. Obvious abdominal tuck. Thin neck and |
| | | narrow waist. |
| 2 | Underweight | Backbone and ribs easily palpable. Minimal fat |
| | | covering. Minimal waist when viewed from above. |
| | | Slightly tucked abdomen. |
| 3 | Ideal | Ribs palpable but not visible. Slight waist observed |
| | | behind ribs when viewed from above. Abdomen |
| | | tucked, flank folds present. |
| 4 | Overweight | Ribs palpable with slight excess of fat covering. |
| | | Abdomen slightly rounded, flank concave. Flank |
| | | folds hang down with moderate amount of fat - jig- |
| | | gle noted when walking. |
| 5 | Obese | Ribs and backbone not easily palpable under a |
| | | heavy fat covering. Abdomen rounded; waist barely |
| | | visible to absent. Prominent flank folds that sway |
| | | from side to side when walking. |

TABLE 2
Treatment group codes

T × 1 = 3% Free fatty acids
T × 2 = 3% Ethyl esters of fatty acids
T × 3 = 0.3% Lotus leaf extract
T × 4 = Control Diet

TABLE 3
Whole Body Composition (Absolute Data)

| | | | | | | | Lean + BM | | | | |
|---|---|---|---|---|---|---|---|---|---|---|---|
| | | Area | BMC^{a} | BMD^{a} | Fat | Lean | C | Total | Fat | Lean | BMC |
| | | cm² | (g) | (g/cm³) | (g) | (g) | (g) | (g) | (%) | (%) | (%) |
| Day 0 | T × 1 | 402.07 | 263.95 | 0.65 | 5772.09 | 9840.21 | 10104.15 | 15876.27 | 35.74 | 62.58 | 1.67 |
| | T × 2 | 395.54 | 253.62 | 0.64 | 5956.12 | 9667.31 | 69164.51 | 15877.02 | 36.77 | 61.63 | 1.61 |
| | T × 3 | 408.44 | 262.16 | 0.64 | 5897.71 | 9827.01 | 10089.17 | 15986.89 | 36.21 | 62.14 | 1.65 |
| | T × 4 | 387.81 | 252.69 | 0.65 | 6109.95 | 9882.50 | 10135.17 | 16245.15 | 36.75 | 61.67 | 1.58 |
| Day 84 | T × 1 | 387.39 | 257.27 | 0.66 | 4109.94 | 9607.27 | 9864.54 | 13974.49 | 29.22 | 68.94 | 1.84 |
| | T × 2 | 362.26 | 229.74 | 0.63 | 4723.12 | 9837.15 | 10066.89 | 14790.01 | 31.44 | 66.99 | 1.57 |
| | T × 3 | 352.09 | 230.55 | 0.65 | 4076.82 | 10353.58 | 10584.13 | 14660.96 | 27.39 | 71.04 | 1.57 |
| | T × 4 | 360.36 | 239.68 | 0.66 | 4390.91 | 9882.87 | 10122.55 | 14513.44 | 29.59 | 68.75 | 1.67 |

^{a}BMC = bone mineral content; BMD = bone mineral density.

TABLE 4
Whole Body Composition (Relative Data)

| | Area | BMC^{a} | BMD | Fat | Lean^{a} | Lean + BMC | Total | Fat | Lean | BMC |
|---|---|---|---|---|---|---|---|---|---|---|
| | (%) | (%) | (%) | (%) | (%) | (%) | (%) | (%) | (%) | (%) |
| T × 1 | −3.98 | −3.06 | 1.15 | −28.22 | −2.34 | −2.38 | −11.75 | −18.59 | 10.84 | 10.16 |
| T × 2 | −7.99 | −8.58 | −0.58 | −18.59 | 2.06 | −4.69 | −6.56 | −13.43 | 9.61 | −2.39 |
| T × 3 | −13.75 | −12.13 | 2.11 | −29.54 | 5.83 | 5.34 | −7.52 | −24.21 | 14.86 | −4.57 |
| T × 4 | −6.92 | −5.33 | 2.07 | −26.95 | 0.40 | 0.23 | −10.37 | −18.73 | 12.28 | 5.59 |

TABLE 5
Average Body Condition Scores

| | Day 0 | Day 84 | % Change |
|---|---|---|---|
| T × 1 | 4.1 | 3.3 | −19.5 |
| T × 2 | 4.0 | 3.7 | −7.5 |
| T × 3 | 3.9 | 3.6 | −7.7 |
| T × 4 | 4.2 | 3.6 | −14.3 |

All documents cited herein are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for promoting weight control in a companion animal comprising orally administering one or more non-glyceryl fatty acid derivatives of C₁₇ or greater fatty acids.

2. The method of claim 1 wherein the companion animal is selected from the group consisting of dogs, cats, and combinations thereof.

3. The method of claim 2 wherein the derivatives are administered as a supplement separate from the companion animal's food.

4. The method of claim 2 wherein the derivatives are administered with the companion animal's food.

5. The method of claim 2 wherein the derivatives are incorporated into the companion animal's food.

6. The method of claim 5 wherein the food comprises, on a dry matter basis by weight, at least about 0.5% non-glyceryl fatty acid derivatives, about 15% to about 55% protein, and about 9% to about 35% dietary fat.

7. The method of claim 6 wherein the food comprises, on a dry matter basis by weight, at least about 1% non-glyceryl fatty acid derivatives.

8. The method of claim 7 wherein the food comprises, on a dry matter basis by weight, about 1% to about 13% non-glyceryl fatty acid derivatives.

9. The method of claim 8 wherein the fatty acids in the fatty acid derivatives contain from 17 to 24 carbons and from 0 to 5 carbon-carbon double bonds.

10. The method of claim 9 wherein the fatty acids in the fatty acid derivatives are selected from the group consisting of heptadecanoic acid, stearic acid, arachidic acid, behenic acid, lignoceric acid, cerotic acid, oleic acid, cis-11-octadecenoic acid, linoleic acid, alpha-linoleic acid, gamma-linolenic acid, eicosapentaenoic acid, docasahexaenoic acid, arachidonic acid, erucic acid, and mixtures thereof.

11. The method of claim 10 wherein the fatty acids in the fatty acid derivatives are selected from the group consisting of linoleic acid, oleic acid, and mixtures thereof.

12. The method of claim 10 wherein the derivatives are free fatty acids.

13. The method of claim 10 wherein the fatty acid derivatives are C₁-C₆ alkyl fatty acid esters.

14. The method of claim 13 wherein the fatty acid derivatives are selected from the group consisting of ethyl oleate, ethyl linoleate, and mixtures thereof.

15. The method of claim 10 wherein at least about 100 mg of non-glyceryl fatty acid derivatives is administered per kg body weight per day.

16. The method of claim 10 wherein the fatty acid derivatives do not cause the companion animal to reduce food consumption.

17. A dietary composition for promoting weight control in a companion animal, the composition comprising, on a dry matter basis by weight, at least about 0.5% of one or more non-glyceryl derivatives of C₁₇ or greater saturated or monounsaturated fatty acids, about 15% to about 55% protein, and about 9% to about 35% dietary fat.

18. The composition of claim 17 comprising, on a dry matter basis by weight, about 1% to about 13% non-glyceryl fatty acid derivatives.

19. The composition of claim 18 wherein the fatty acid derivatives are free fatty acids.

20. The composition of claim 18 wherein the fatty acid derivatives are C₁-C₆ alkyl fatty acid esters.

21. The composition of claim 20 wherein the non-glyceryl fatty acid ester is selected from the group consisting of ethyl oleate, ethyl linoleate, and mixtures thereof.

22. The composition of claim 18 further comprising a component selected from the group consisting of corn meal, sorghum, barley, fish meal, dried beet pulp, dried egg product, brewer's yeast, chicken, beef, lamb, and mixtures thereof.

23. The composition of claim 18 further comprising a source of protein selected from the group consisting of chicken, turkey, beef, fish, lamb, and mixtures thereof.

24. The composition of claim 23 which is a dry kibble.

25. A method for promoting weight control in a companion animal comprising orally administering lotus leaf extract.

26. The method of claim 32 wherein the companion animal is selected from the group consisting of dogs, cats, and combinations thereof.

27. The method of claim 26 wherein the administering lotus leaf extract promotes a decrease or maintenance of fat and an increase or maintenance of lean body mass in the companion animal.

28. The method of claim 26 wherein the lotus leaf extract does not cause the companion animal to reduce food consumption.

29. The method of claim 29 wherein wherein the lotus leaf extract is incorporated into the companion animal's food and wherein the food comprises, on a dry matter basis by weight, at least about 0.05% lotus leaf extract.

30. The method of any one of claim 29 wherein the food comprises, on a dry matter basis by weight, from about 15% to about 55% protein and from about 9% to about 35% dietary fat.

32. A dietary composition for promoting weight control in a companion animal, the composition comprising, on a dry matter basis by weight, at least about 0.05% lotus leaf extract.

33. The composition of claim 32 further comprising, on a dry matter basis by weight, from about 15% to about 55% protein and from about 9% to about 35% dietary fat.

34. The composition of claim 33 further comprising a source of protein selected from the group consisting of chicken, turkey, beef, fish, lamb, and mixtures thereof.

35. The composition of claim 34 which is a dry kibble.

36. The composition of claim 35 comprising, on a dry matter basis by weight, about 0.1% to about 2% lotus leaf extract.

37. A method for promoting weight control in a human comprising orally administering lotus leaf extract, wherein the lotus leaf extract does not cause the human to reduce food consumption.

38. The method of claim 37 wherein the administering lotus leaf extract promotes a decrease or maintenance of fat and an increase or maintenance of lean body mass in the human.
